(19) United States
(12) Patent Application Publication
(10) Pub. No.: US 2014/0018638 A1
(43) Pub. Date: Jan. 16, 2014

(54) Persuasive Sensing Technology: A New Method to Monitor, Track and Assist Older Adults Suffering from Type-2 Diabetes

(71) Applicant: Samir Chatterjee, Rancho Cucamonga, CA (US)

(72) Inventor: Samir Chatterjee, Rancho Cucamonga, CA (US)

(21) Appl. No.: 13/935,534

(22) Filed: Jul. 4, 2013

Related U.S. Application Data
(60) Provisional application No. 61/668,115, filed on Jul. 5, 2012.

(51) Int. Cl. A61B 5/00; A61B 5/145; A61B 5/11

(52) U.S. Cl. CPC A61B 5/002; A61B 5/11; A61B 5/4806; A61B 5/14532; A61B 5/0022; A61B 5/486; A61B 5/72; A61B 5/4884
USPC 600/301

(57) ABSTRACT

This invention relates generally to a method and an apparatus for remote health monitoring of activity of daily living of patients with Type-2 Diabetes. In particular, it relates to a method of capturing vital signs data (Blood-Glucose values, weight) from medical devices, a variety of ambient sensors (on/off sensors, motion sensors, pressure-pad sensors), a body-wearable sensor (arm-band with accelerometer, stress and sleep measurements), and food/diet information uploaded via a web-based interface, then storing all the data in real-time on a secured server, and then processing it to generate short text-messages delivered via cell-phones, iPads or other ambient displays. These messages are designed to be persuasive in nature to alter human behavior. The system can also generate from the data health newsletter that is customized for the patient to educate, inform and empower the patient.

### CROSS-REFERENCE TO RELATED APPLICATION

| Application Number | Filing Date | Patent Number | Issue Date |
|---|---|---|---|
| 61/668,115 | Jul. 5, 2012 | | |
| Current US Class: | 705/2 | | |

| | |
|---|---|
| Current International Class: | G06Q 10/00 (20060101); G06Q 50/00 (20060101) |
| Current CPC Class: | A61B 5/0002 (20130101); G06F 19/3418 (20130101); G06F |
| | 19/3493 (20130101) |

### CLAIM OF PRIORITY

This application claims priority of the U.S. Provisional Application No. 61/668,115 filed Jul. 5, 2012 which is incorporated herein by reference.

### FIELD OF THE INVENTION

This invention relates generally to a method and an apparatus for remote health monitoring of activity of daily living of patients with Type-2 Diabetes. More specifically, this is a method and apparatus for improving the daily activity of elderly people with type 2 diabetes by tracking these elderly people living habits and intervening with text messages and newsletters to improve activity levels.

### BACKGROUND OF THE INVENTION

Diabetes mellitus is the most common and serious chronic disease facing the entire global population. In the United States, there are nearly 26 million Americans with diabetes, 30% of which are aged 65 and older [1]. Diabetes is a chronic disease, which if unchecked leads to acute and long-term complications and ultimately death. Our older adult population often lacks the cognitive resources to deal with the daily self-management regimens. Many unpaid family members are caring for them today but this is unsustainable.

When we consider the state of California (our residence), we see an impending crisis that is emerging. According to the 2010 census, there were 4.3 million Californians 65 and older (referred to as older adults from now on) accounting for about 11% of the total state population. More specifically, of the 4.3 million, about 86% are age 65-84 and about 14% are age 85 and older [2, 26]. Exploring further we find that 58% of older adults have high blood pressure, about 21% have been told that they have diabetes. 37% of older adult Californians are classified as overweight and about 22% are classified as obese [2]. The most worrisome statistic is that the number of Californians age 65 and older is projected to increase by 100% from 2010 to 2030 (7.75 million as the Baby Boomer generation turns 65 years old) [27].

Diabetes is a chronic disease characterized by a sustained elevated blood glucose level, caused by a reduction in the action of insulin secretion where related metabolic disturbances generate severe, acute and long-term complications that are responsible for premature death and disability [10]. The World Health Organization projects that diabetes deaths will increase by more than 50% in the next ten years without urgent action. Most notably, diabetes deaths are projected to increase by over 80% in low-middle income countries between 2006 and 2015 [17]. The costs of caring for this disease are astronomical and are estimated to exceed more than $24 billion in California and $174 billion nationally [1, 2, 27].

These older adults are receiving long-term care services which are being provided in a variety of settings including one's home (home care), in the community (e.g., adult day care), in residential settings (e.g., assisted living or board and care homes), or in institutional settings (e.g., intermediate care facilities or nursing homes). In 2007, 4 million Californians served as unpaid caregivers to an adult or child. The majority of unpaid caregivers in California (85%) are family members and almost half of unpaid caregivers are caring for a parent. In 2010, approximately 42% of Californians 65 and older are living alone.

This situation is unsustainable. While diabetes is a dangerous disease, it can be managed if the patient can adhere to recommended ADA self-management guidelines [10]. Regularly measuring blood-sugar levels, staying physically active, watching diet and calorie in-take, and not forgetting to take medications and insulin can help to manage the disease. Yet our older adult population lacks the cognitive resources and problem-solving skills to deal with the daily regimens. The older adult population has difficulty in self-management because their memory becomes weak with age, they forget often and the daily stress of life can be overwhelming for them. Here we propose new ways of addressing these problems with the help of mobile wireless information technology solutions.

Medical devices, information technology and mobile communications have started to converge; this has the potential to revolutionize healthcare in the home [14, 28].

At-home healthcare can help address the social and financial burdens of an aging population. At the same time the technology can support the network of care-givers such as family members, neighbors, and friends with new and innovative ways to monitor the wellbeing of older people, increase the levels of communication with the older person and to enable rapid response to emergency situations. The present invention is aimed at lowering care-giver burden while enhancing the patient's quality-of-life.

Using wireless sensor networks within the home can help to remotely monitor activity of daily living (ADL) [14, 19]. Such data if mined properly can identify health patterns which can then be used to send effective reminders and feedback [13, 18]. Mobile phones are an ideal platform for sending feedback to diabetes patients because they are ubiquitous, low-cost, reliable, real-time, and versatile; and unlike most technologies, actually enjoy greater usage amongst racial and ethnic minorities. Mobile phones can be self-management tool that can help individuals remember various health-related activities and record them, and also help others in their personal wellness ecosystem to review ongoing health patterns and respond quickly to changes in health status [11, 12].

In this patent proposal, we discuss the design and implementation of a wireless sensor network system within the home environment that captures activity of daily living. We introduce a novel idea called “persuasive sensing technology” or PST. Basically PST involves capturing relevant activity data from a variety of commercially available sensors, and using algorithms to process contextual data and send text messages to subjects to alter their human behavior. We mine the sensor data and provide feedback via SMS/text (daily) and a tailored newsletter (weekly). Results and findings from two case studies show a lot of promise for this technology.

### BRIEF SUMMARY OF THE INVENTION

The Persuasive Sensing Technology (PST) that we propose here is a remote health monitoring system that works with FDA approved medical devices and a variety of body-wearable and ambient sensors. It combines data from several ambient sensors as well as body-wearable sensors to provide a rich data set of activity of daily living as it relates to diabetes self-management. Our algorithm then mines the data and provides context-based text messages along with a tailored health newsletter to help change behavior. The messages are transmitted as short-text messages (SMS) that can displayed over cell-phones and/or smartphones. The same messages can also be displayed on Android tablets, iPads or even Television sets. The messages are persuasive in nature and aimed to empower the patient to better manage their chronic condition.

It is therefore an object of the present invention to introduce a method and apparatus for improving the daily activity of elderly people with type 2 diabetes by tracking these elderly people living habits and intervening with text messages and newsletters to improve activity levels.

### BRIEF DESCRIPTION OF THE DRAWINGS

FIG. 1 is a flow diagram of a home with installed sensors of the present invention.

FIG. 2 is an architectural diagram showing an implementation within a home of data capture and processing

FIG. 3 is a flow diagram of an experimental design for intervention of the present invention.

FIG. 4 is a scatter plot graph of a blood glucose level pertaining to the algorithm of the present invention.

FIG. 5 is a scatter plot graph tracking weight over time pertaining to the algorithm of the present invention.

FIG. 6 is a scatter plot graph tracking idle time over time pertaining to the algorithm of the present invention.

FIG. 7 is a scatter plot graph tracking number of steps over time pertaining to the algorithm of the present invention.

### DETAIL DESCRIPTIONS OF THE INVENTION

All illustrations of the drawings are for the purpose of describing selected versions of the present invention and are not intended to limit the scope of the present invention.

The present invention is a method and apparatus for human tracking and intervention. The present invention is comprised of the following components: a wireless sensor network (WSN), ambient sensors, device-level sensors, body-wearable sensors, short message service (SMS) interventions, newsletter interventions, a novel persuasive messaging algorithm, eligible subjects of the present invention, and experimental design.

The present invention comprises a wireless sensor network as shown in the following: Any at-home healthcare solution must detect and respond to the activities and/or characteristics of the older person. A network of sensors (worn, carried, or environmental) is an ideal technology platform for detecting and responding to health-relevant parameters such as movement, sleep, weight, physiological data and social activity [14]. In designing the present invention, the following key principles were kept in mind throughout the process:
This is a healthcare problem, not a technology problem. At the center is the patient, not the technology. That also means as the experiment progresses, the research team must adapt based on patient's feedback.
The simpler the technology, the better. Patients must comprehend what is being sent as feedback.
Wireless Sensor Networks (WSNs) for healthcare are mission-critical; reliability is of paramount importance.
The daily feedback persuasive messages must be kept fresh and not boring so that patient is eager to receive them and learn how to change his/her behavior.
The WSN must work in the home, not just in the lab.

A WSN device is a packaged data collecting or actuating component, which includes a sensor and/or actuator, a radio stack, an enclosure, an embedded processor, and a power delivery mechanism [14]. The sensor interacts with the environment and sends an appropriate signal (analog or digital) to the embedded processor (also called microcontroller unit). A research team can use but is not limited to using the Iris Mote technology developed by Intel and UC Berkeley labs. The mote hardware platform consists of a microprocessor and radio chip (MPR: Mote Processor Radio Board).

Sensors connect directly to the mote processor radio boards via various interfaces. This combination gives the mote the ability to sense, compute and communicate. The mote enables raw data collected by the sensors to be analyzed in various ways before sending it to an aggregator (in our case a laptop) that the research team places within the home. The aggregator then uploads daily activity data to the cloud through secured channels via the Internet.

The present invention comprises an ambient sensor as shown in the following: The ambient sensor is a simple on/off switch that detects open/close of garage door (through which subjects leave home), detects the back porch door for outdoor access. An infrared analog sensor is used to detect presence in the bedroom. A pressure pad sensor (such as an apparatus sold by Colonial Medical) is then placed in the couch in the living room in front of TV. Simple on/off switches are used to detect opening and closing of medication cabinet and the cabinet containing insulin. A photo sensor is connected to the TV to detect television viewing.

The present invention comprises a device-level sensor as shown in the following: The device-level sensor is a blood glucose monitor device can connect easily to the laptop via USB and can upload blood glucose values daily. A wireless weight machine (such as an apparatus manufactured by Tanita Corporation) sends weight values via Bluetooth are placed in the family room.

The present invention comprises a body-wearable sensor as shown in the following: The commercial body-wearable sensor in the present invention is an armband (such as an apparatus manufactured by BodyMedia Inc.) which is given to the patient to wear 24-hours a day. This multi-sensor senses the number of steps walked, quality of sleep, skin temperature, and many other physiological parameters of the subject. Data from the body-wearable sensor is uploaded to the cloud by connecting it to USB port for less than five minutes daily.

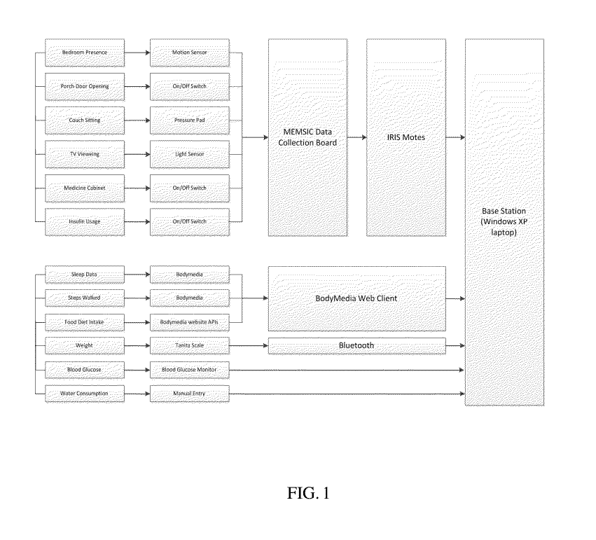

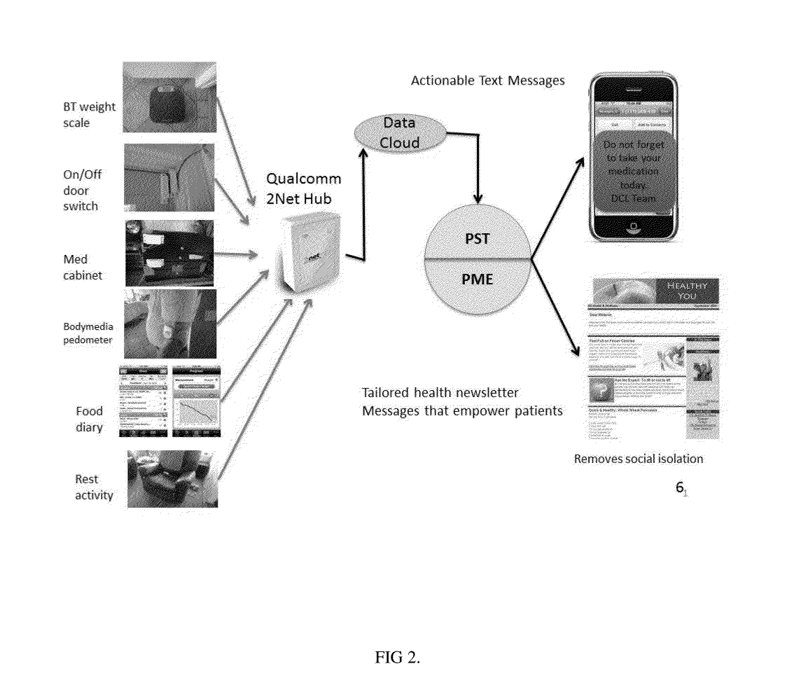

The subject is shown how to log into a health website (such as a website operated by BodyMedia) where he/she can input diet/nutrition information. The system of the present invention then fetches daily diet data and which is then computed with total calories consumed. The support team also provides the patient with bottled water and asks them to drink that during the course of the experiment. This is a simple way for us to monitor water intake. The overall sensor-based data collection apparatus scheme is shown in FIG. 1 and an actual implementation of the same within the home is shown in FIG. 2.

In reference to FIG. 2, a seat in the living room has a pressure pad installed, the television in the living room has an infrared sensor installed, the family room has a laptop, the family room has an insulin apparatus, the family room has a wireless scale, the bedroom door has an infrared sensor, the garage door has an on/off switch sensor, and the porch has an on/off switch sensor.

The present invention comprises SMS and newsletter interventions as shown in the following: Patients with type 2 diabetes can manage their chronic conditions by following certain recommended strategies.

Prevention strategies for Type 2 diabetes include:
Losing weight and keeping Body Mass Index (BMI) under control
30 minutes or more of exercise or physical activity (brisk walking every day is fine)
Developing a low calorie and low fat diet. Nutrition guidelines include recommendations for a diet rich in whole grains, fruits and vegetables.
Taking necessary medications (including insulin) and measuring blood sugar levels regularly.

Most elderly patients cannot adhere to these regimens due to lower cognitive abilities and lack of resources to maintain the lifestyle. Hence with the present invention, it is now possible to help these patients.

The intervention strategy developed here interacts with the patient in two distinct ways.
An automated system sends daily Short Message Service (SMS) to a subject's cell phone. These messages are persuasive and target behavior change in the subject. We try to restrict around 1-3 SMS daily so as not to overwhelm the patient.
A tailored newsletter that summarizes healthy living parameters is presented to subject once a week and is jointly read by family member or someone from the care-giver team.

The present invention comprises an algorithm as shown in the following:

Note interventions (through the prototype persuasive sensing system) are aimed to engage patients in diabetes self-management through interactive SMS and newsletter approaches. It is important to ensure that daily text messages that are sent to the subject are fresh and relevant. Each day the subjects receives up to 3 text messages that are delivered to them over an LG smart phone (subject 1) and an iPhone (subject 2). Prior research has shown the efficacy of telephone reminders [29] and technological cues. However, our system sends feedback based on the actual subject's daily behavior, which is much more targeted and context relevant.

Based on processing the last 24-hour sensor data, we design specific tailored messages to be sent out to the subjects. The phrasings of the messages are carefully done following guidelines from health promotion literature. For example, when the research team measures the physical activity of the subject, the research team sets a target goal for daily number of steps walked. If the actual number is below the target, the research team sends motivational messages while if actual number exceeds the research team sends rewarding type of messages. Details are shown in Tables 1 through 5.

Physical activity is measured by the number of steps recorded by the body-wearable sensor on the subject. Table 1 is an example of the SMS interventions that vary depending on the body-wearable sensor reading. The subject has self-selected goal for example 8000 steps per day. This goal varies with each subject. The subject receives a praising intervention or an inspirational intervention depending on the subject's performance. Table 1 shows possible feedback interventions.

Number of steps taken is represented by n in Table 1.

TABLE 1
Messaging for number of steps and physical activity

| Case | Monday | Tuesday | Weds | Thursday | Friday | Sat | Sunday |
|---|---|---|---|---|---|---|---|
| n >= | Great Job! | You have exceeded | You are | You are a super | You have | Your | Great Job. |
| 8000 | Keep up the | your goal. | doing very | hero. You have | exceeded | physical | Enjoy the |
| | good work. | Congratulations. | well. Keep | exceeded your | your goal. | activity trend | Sunday with |
| | | | it up! | goal again. | Super job! | is rising. | friends and |
| | | | | | | Keep | family. |
| | | | | | | moving. | |
| N < | Don't give up | Don't give up on | Try a little | You fell short of | Never say | Try some | It is a beautiful |
| 8000 | on physical | physical activity. | harder to | your goal. Don't | never. You | brisk when | day. Go out |
| | activity. Try | Have you taken the | reach your | worry. Try to | can do it. | you are in the | and do brisk |
| | walking a mile | stairs? | goal of | walk a mile after | | parking lot. | walking for 30 |
| | each day. | | 8000 steps? | dinner. | | | mins. |

Caloric intake is measured by the health website that the user regularly fills out.

Table 2 is an example of the SMS interventions that vary depending on the health website input. The subject has goal of 2500 calories per day. This goal stays static with each subject. The subject receives a praising intervention or an inspirational intervention depending on the subject's performance. Table 2 shows possible feedback interventions.

Caloric intake is represented by c in Table 2.

TABLE 2
Messaging plan for food and calorie intake

| Case | Monday | Tuesday | Weds | Thursday | Friday | Sat | Sunday |
|---|---|---|---|---|---|---|---|
| c <= | Your | Great job. | Enjoy green | Your diet calorie | Very well done. | Your diet | Show |
| 2500 | careful diet | You are | vegetables and | intake is under | If you can walk | trend is | calorie |
| | is going to | watching your | salads and you | control. | 5000 extra steps, | looking very | graph for |
| | help you | diet and will | are on your way | Congratulations! | then treat | good. Keep it | past 6 |
| | reduce | see results | to lose weight. | | yourself a | up. | days. |
| | weight. | soon. | | | starbucks | | |
| | | | | | frappacino. | | |
| c > | Try to eat | Avoid | Try 10 baby | Did you know that | Have you tried | Add variety | Choose |
| 2500 | reduced | takeaways and | carrots and a | obesity is one of the | low calorie | of colorful | low-fat |
| | portions | snack foods | tablespoon of | leading causes of | drinks such as | vegetables to | dairy |
| | today! | that are high | fat-free dressing | death in this country? | diet sprite? | your meal | foods and |
| | | in fat. | for a 100- | | | today. | lean meat. |
| | | | calorie snack. | | | | |

Blood glucose value is measured by blood glucose monitor that is attached to the user's website. Table 3 is an example of the SMS interventions that vary depending on whether or not the subject measures his/her blood glucose. The subject has goal of measuring his/her blood glucose every interval that is suggested by their doctor. This goal stays static with each subject. The subject receives a praising intervention or an inspirational intervention depending on the subject's performance. Table 3 shows possible feedback interventions.

Blood glucose tests are either taken or forgotten tests represented by present or absent in Table 3.

TABLE 3
Messaging plan for blood-glucose values

| BG | | | | | | | |
|---|---|---|---|---|---|---|---|
| value | Monday | Tuesday | Weds | Thursday | Friday | Sat | Sunday |
| present | Blood sugar is | Keep | While fasting | BG values | Normally, a | Your BG trends | Keep |
| | the main | monitoring your | your BG | measured | hormone called | are looking in | monitoring |
| | source of | BG levels every | level should | after meals | insulin helps | the right | your BG levels |
| | energy for our | day. | be 130 | typically | sugars in the | direction. | every day. |
| | organs. Keep | | mg/DL | show higher | blood enter | | |
| | physically | | | values. | your body | | |
| | active to help | | | | cells, where | | |
| | break food into | | | | they are used | | |
| | sugar. | | | | for energy. | | |
| absent | You must | Did you forget | Knowing | Did you | Did you forget | Missing BG | Did you know |
| | measure your | to take your | your BG | know that | to measure | measurement | that some |
| | BG levels | blood-glucose | levels is | some people | your BG | can hurt keeping | people |
| | every day. | measurement? | important as | measure | levels? | track of your | measure blood |
| | | | it produces | blood | | daily BG | glucose even |
| | | | the necessary | glucose even | | values. | two or three |
| | | | energy in | two or three | | | times a day? |
| | | | your body. | times a day? | | | |

Sedentary activity is measured by ambient sensors placed around the subject's house. Table 4 is an example of the SMS interventions that vary depending on whether or not the subject stays active during the day. The subject has goal staying sedentary for less than 5 hours a day. This goal stays static with each subject. The subject receives a praising intervention or an inspirational intervention depending on the subject's performance. Table 4 shows possible feedback interventions.

Shown is an example of how messages were varied for physical activity (Table 1), diet and calorie intake (Table 2), blood-glucose values (Table 3) and sedentary or idle activity (Table 4). The customized newsletter is a PDF file that is about 4-5 pages and carefully summarizes the details of the subject's weekly performance. The newsletter is read together by the subject and one of our team members.

TABLE 4
Messaging plan for sedentary activity

| Case | Monday | Tuesday | Weds | Thursday | Friday | Sat | Sunday |
|---|---|---|---|---|---|---|---|
| data < | You are | Being sedentary | Stay active. | Well done. You | Your | Your sedentary | Enjoy the |
| 5 | moving well. | does not help | This will lower | are moving | physical | time trend is | Sunday by |
| | Sitting idle | your blood | your BG | throughout the | activity is | really | doing some |
| | does not help | sugar levels to | levels. | day. | helping you | improving. | household |
| | to fight | come down. | | | lower your | | chores. |
| | diabetes. | | | | BG levels. | | |
| data > | You have been | Don't give up | Avoid being | Take some rest | Avoid TV | Your sedentary | It is a beautiful |
| 5 | slightly more | on physical | stationary. | but try not to be | today. | time trend can | day. Call a |
| | idle today. Try | activity. Avoid | Take a brisk | too idle at any | Instead walk | be improved by | friend or |
| | to do some | sitting in one | walk whenever | point of the | a mile. | being less idle. | neighbor and go |
| | activity within | place for more | you can. | day. | | | for a brisk walk |
| | the home. | than 30 mins. | | | | | outside. |

The present invention comprises specific subjects as shown in the following:

The research team obtains approval from a university Institutional Review Board (IRB). After IRB approves the research team to proceed, the research team distributes announcements to recruit subjects via hospitals, diabetes clinics and through personal contacts. The basic eligibility criteria that the research team includes in recruitment efforts are:
Subject must have Type 2 diabetes
Age can be between 45-85
Gender and race—no preference
Have familiarity with cell phone and texting
Have a broadband internet connection at home

The research team receives prospective candidates who expressed interest. From the pool the research team can select a plurality of subjects. In this particular case the first subject was an 82 year old white male who is retired and lives in the Vista community near San Diego. He has type 2 diabetes, and also a few other health problems. He agreed to the consent form and the researchers started our project implementation.

In this particular case the second subject was a 60 year old female patient who lives in San Diego. She also has type-2 diabetes, hypertension and is obese.

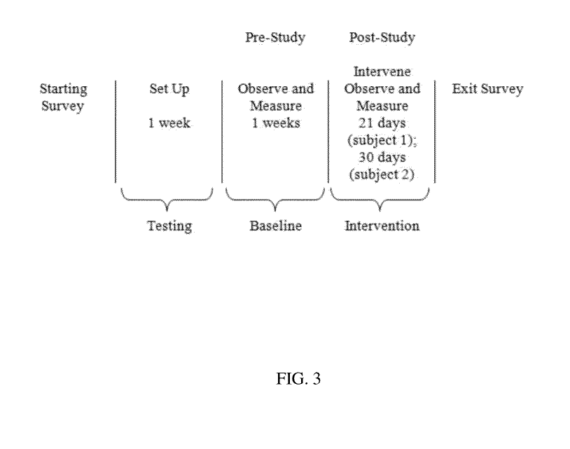

The research team specifically designs a pre-post type of intervention (see FIG. 3).

The research team also does a sequential implementation. In this particular case, the first implementation was at the male subject's home which started on Oct. 18, 2011 and ended on Nov. 25, 2011. In this particular case, the second home implementation was the female subject which started on January 2 and ended on March 1. The research team encountered certain unnecessary delays with our second subject. All sensors and other equipment's were removed from subjects home after the intervention. The research team thanked the subjects and gave them a token honorarium of for participation.

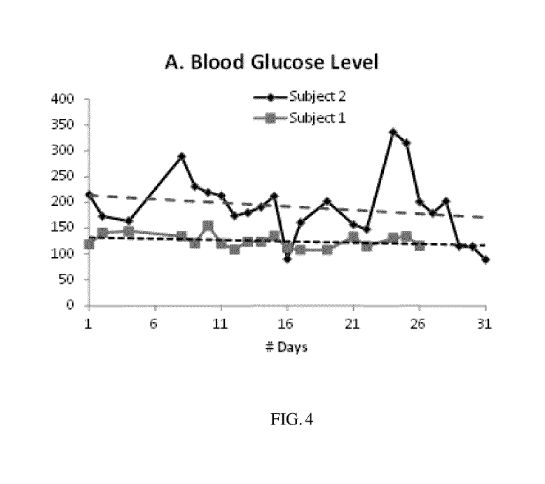

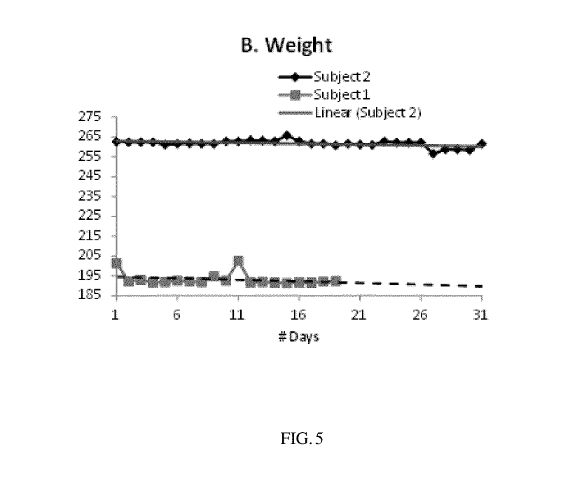

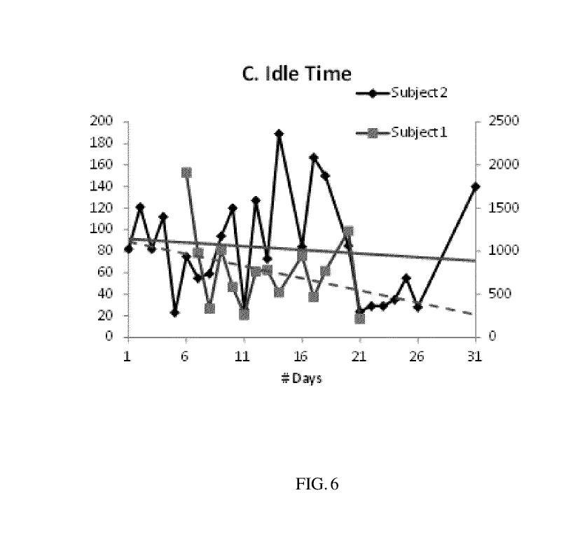

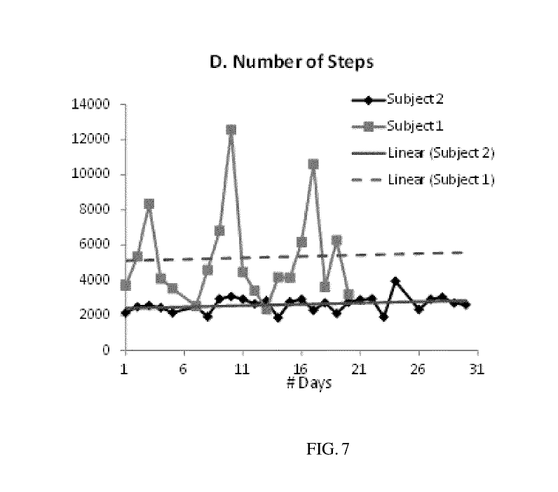

In this particular case, the trend lines for BG-levels (FIG. 4) for both the subjects show a gradual decline. That is a good trend. In fact the research team had asked the subjects to get their HbAlc (considered a 90-day average of blood sugar) before and after the intervention. Subject #1 had a drop from 12.8% to 6.6% HbAlc which is a significant improvement (50% decreases). In subject #2 HbAlc went down from 8.9% to 8.5% which is also a positive result. It is easy to see that subject #2 had greater daily fluctuation of her BG-levels. The weight (FIG. 5) and idle-time (FIG. 6) trends also show a decline. FIG. 7 shows that the trend in number of steps walked (which reflects physical activity) is increasing. The research team's daily text messages and newsletters help to alter the subject's behavior.

The research team designs and builds an in-home activity monitoring system using ambient sensors and body-wearable sensors. Using a pre-post experiment method, the subject(s) receives daily text messages based on his/her behavior the previous day.

These persuasive messages use strategies such as motivate, praise, guilt or reward to encourage positive behavior change. The subject also receives a tailored health newsletter at the end of each week that summarized various physiological and biological parameters. In this particular case, subject 1 showed tremendous improvement in HbAlc levels which dropped from 12.9% to 6.6% when measured after experiment. In this particular case, subject 2's HbAlc went down from 8.9% to 8.5%.

The research team then conducts a post-experiment exit survey in which self-efficacy is assessed using an adapted version of Sarkar et al., diabetes self-efficacy (DSE) scale [30]. The DSE scale is a reliable, validated 4-item instrument that assesses patients' perceived competence in diabetes self-management. In this particular case, results show (Table 5) that subjects' ability to manage diabetes is improving. It also indicates that their quality of life is improving as well. In reference to Table 5, subjects feel that they are confident in managing their diabetes, capable of handling their diabetes, able to do their own routine diabetes care, and able to meet the challenges of controlling their diabetes.

TABLE 3
Diabetes DES exit survey results

| | Subject 1 (82 year old male) | | Subject 2 (60 year old female) | |
|---|---|---|---|---|
| | Pre-Persuasive- | Post Persuasive- | Pre-Persuasive- | Post Persuasive- |
| | sensing care | sensing care | sensing care | sensing care |
| I feel confident in my | agree | agree | agree | Strongly agree |
| ability to manage my | | | | |
| diabetes | | | | |
| I feel capable of | agree | agree | agree | agree |
| handling my diabetes | | | | |
| I am able to do my own | agree | Strongly agree | agree | Strongly agree |
| routine diabetes care. | | | | |
| I am able to meet the | neutral | neutral | neutral | agree |
| challenge of controlling | | | | |
| my diabetes. | | | | |

The research team's persuasive-sensing system with specialized messaging algorithms designed for diabetic patients is a significant contribution towards achieving healthy lifestyles for older patients who are suffering from this deadly disease. Such systems relieves care-giver burden and helps patients to better self-manage their chronic condition. In the above two case studies, the researchers present a novel idea. Our next step would be to scale up to 100 homes and conduct a detailed Random Control Trial experiment.

The present invention lowers a subject's 90-day average of blood sugar, lowers weight, lowers idle-time, and increases physical activity. The research team's daily text messages and newsletters help to alter the subject's behavior. This effectively helps elderly people with type 2 diabetes lead much healthier lifestyles.

Although the invention has been explained in relation to its preferred embodiment, it is to be understood that many other possible modifications and variations can be made without departing from the spirit and scope of the invention as hereinafter described.

### ACKNOWLEDGEMENT

This project was partially supported by an EAGER grant from the National Science Foundation CNS Award #1048366.

### REFERENCES CITED

U.S. Patent Documents

| | | |
|---|---|---|
| 7,399,276 | July 2008 | Brown et al. |
| 8,024,201 | September 2011 | Brown, Stephen J. |

### Other References

[1] Centers for Disease Control and Prevention, “National diabetes fact sheet: national estimates and general information on diabetes and pre-diabetes in the United States, 2011,” Atlanta, Ga.: U.S. Department of Health and Human Services, Centers for Disease Control and Prevention, 2011.
[2] California Diabetes Program, Diabetes Information Resource Center, “California diabetes fact sheet,” California Department of Public Health, University of California San Francisco, 2011.
[3] H. Saydah, J. Fradkin, and C. C. Cowie, “Poor control of risk factors for vascular disease among adults with previously diagnosed diabetes,” JAMA: The Journal of the American Medical Association, vol. 291, no. 3, pp. 335-342, January 2004.
[4] J. B. Saaddine, B. Cadwell, E. W. Gregg, M. M. Engelgau, F. Vinicor, G. Imperatore, and K. M. Narayan, “Improvements in diabetes processes of care and intermediate outcomes: United states, 1988-2002.” Ann Intern Med, vol. 144, no. 7, pp. 465-474, April 2006.
[5] A. Bethel, F. A. Sloan, D. Belsky, and M. N. Feinglos, “Longitudinal incidence and prevalence of adverse outcomes of diabetes mellitus in elderly patients.” Arch Intern Med, vol. 167, no. 9, pp. 921-927, May 2007.
[6] F. A. Sloan, M. A. Bethel, D. Ruiz, A. M. Shea, A. H. Shea, and M. N. Feinglos, “The growing burden of diabetes mellitus in the US elderly population.” Archives of internal medicine, vol. 168, no. 2, pp. 192-199, January 2008.
[7] J. M. Schectman, M. M. Nadkarni, and J. D. Voss, “The association between diabetes metabolic control and drug adherence in an indigent population,” Diabetes Care, vol. 25, no. 6, pp. 1015-1021, June 2002.
[8] M. Heisler, J. D. Faul, R. A. Hayward, K. M. Langa, C. Blaum, and D. Weir, “Mechanisms for racial and ethnic disparities in glycemic control in middle-aged and older Americans in the health and retirement study,” Arch Intern Med, vol. 167, no. 17, pp. 1853-1860, September 2007.
[9] L. Blonde and A. J. Karter, “Current evidence regarding the value of self-monitored blood glucose testing.” Am J Med, vol. 118, no. Suppl 9A, pp. 20-26s, September 2005.
[10] Living with Diabetes: Treat and Care. American Diabetes Association. At http://www.diabetes.org/living-with-diabetes/treatment-and-care/?loc=DropDownLWD-treatment. Last accessed on Mar. 14, 2012.
[11] B. J. Fogg and R. Adler Eds. Texting for Health: A simple powerful way to improve lives. Stanford Persuasive Lab. 2009.
[12] E. Årsand, N. Tatara, G. Østengen, and G. Hartvigsen, “Mobile Phone-Based Self-Management tools for type 2 diabetes: The few touch application,” Journal of Diabetes Science and Technology, vol. 4, no. 2, pp. 328-336, March 2010.
[13] L. Atallah, B. Lo, R. Ali, R. King, and G. Z. Yang, “Real-time activity classification using ambient and wearable sensors,” Trans. Info. Tech. Biomed., vol. 13, no. 6, pp. 1031-1039, November 2009.
[14] T. J. Dishongh and M. McGrath, Wireless sensor networks for healthcare applications. Boston, Mass.: Artech House, 2010.
[15] L. Liu, “Social connections, diabetes mellitus, and risk of mortality among white and African-American adults aged 70 and older: An Eight-Year follow-up study,” Annals of Epidemiology, vol. 21, no. 1, pp. 26-33, January 2011.
[16] C. A. Chesla, “Do family interventions improve health? ” Journal of Family Nursing, vol. 16, no. 4, pp. 355-377, November 2010.
[17] World Health Organization, 10 facts about diabetes. http://www.who.int/features/factfiles/diabetes/en/index.html, 2011.
[18] G. Virone, M. Alwan, S. Dalal, S. W. Kell, B. Turner, J. A. Stankovic, and R. Felder, “Behavioral patterns of older adults in assisted living,” Information Technology in Biomedicine, IEEE Transactions on, vol. 12, no. 3, pp. 387-398, May 2008.
[19] J. A. Stankovic, “Research challenges for wireless sensor networks,” SIGBED Rev., vol. 1, no. 2, pp. 9-12, July 2004.
[20] B. J. Fogg, Persuasive Technology: Using Computers to Change What We Think and Do (Interactive Technologies), 1st ed., Morgan Kaufmann, December 2002.
[21] S. Chatterjee and A. Price, “Healthy living with persuasive technologies: Framework, issues, and challenges,” J Am Med Inform Assoc, vol. 16, no. 2, pp. 171-178, March 2009.
[22] T. Hassan, and S. Chatterjee. “A Sensor Based Mobile Context-Aware System for Healthy Lifestyle Management,” in Persuasive 2008, Oulu, Finland, 2008.
[23] H. Li and S. Chatterjee, “Designing effective persuasive systems utilizing the power of entanglement: Communication channel, strategy and affect,” in Persuasive Technology, ser. Lecture Notes in Computer Science, T. Ploug, P. Hasle, and H. Oinas-Kukkonen, Eds., Berlin, Heidelberg: Springer Berlin/Heidelberg, 2010, vol. 6137, ch. 27, pp. 274-285.
[24] American Diabetes Association (ADA). http://www.diabetes.org/, Accessed on Feb. 13, 2012.
[25] K. Teknomo, K-Means Clustering Tutorials. http:\\people.revoledu.com\kardi\tutorial\kMean\, Accessed on Feb. 16, 2012.
[26] SCAN Foundation Report. Demographic & Economic Characteristics of Aging Californians (Updated). February 2012.
[27] SCAN Foundation Report. Who needs and Uses Long-Term care in California?
[28] Y. Xiao and H. Chen, Mobile Telemedicine: A Computing and Networking Perspective, 1st ed., Boston, Mass., USA: Auerbach Publications, 2008
[29] Dick J J, Nundy S, Solomon M C, Bishop K N, Chin M H, Peek M E. The Feasibility and Usability of a Text-Message Based Program for Diabetes Self-Management in an Urban African-American Population. J Diabetes Sci Technol. 2011 September
[30] Sarkar U, Fisher L, Schillinger D. Is self-efficacy associated with diabetes self-management across race/ethnicity and health literacy? Diabetes Care. 2006; 29(4):823-9.

What is claimed is:

1. A remote health monitoring system, comprising of a) one or more wireless sensor nodes communication to a central hub with either WiFi, Bluetooth, ZigBee protocols; b) a body-wearable sensor device capable of transmitting accelerometer, stress and sleep data; c) a blood-glucose meter capable of transmitting data either through USB cable upload or via Bluetooth; d) a Bluetooth enable weight-scale; e) a plurality of ambient-sensors such as pressure-pads and motion detection; f) a web-based interface via which food/diet information can be uploaded; g) a simple ON/OFF sensor detecting opening and closing of medicine cabinet; whereby (A) all data is captured and stored in a secure server; (B) processed according to a set of rules created based on the goals of the patient; (C) generating feedback using one or more persuasive short-text messages based on context and relevance of data; (D) generating a customized health newsletter that is customized according to the patient; and (E) the intervention comprising of daily text messages and weekly health newsletter to alter human behavior of a diabetic patient.

2. The system according to claim 1, further comprising the step of providing feedback via displays such as iPad, Android devices, Television screens, basic cell-phones or smartphones.

3. The system according to claim 1, further comprising the step of processing data from the devices and sensors and then choosing persuasive messages from a message pool that is especially phrased to motivate, and empower the patient.

4. The system according to claim 1, further comprising the step of generating messages that is based on goals, context values obtained from the patient and the ability of being adaptive in order to either motivate, reward or provide guilt to enable the patient to do better in terms of reaching his/her wellbeing goals.

5. The system according to claim 1, further comprising the step of generating customized health newsletter based on the data in order to educate, inform and guide wellbeing of the patient.

6. The system according to claim 1, wherein said data is transmitted in a secure manner using standard encryption technology.
